(19) United States
(12) Patent Application Publication
(10) Pub. No.: US 2024/0379213 A1
(43) Pub. Date: Nov. 14, 2024

(54) NUTRITIONAL COMPOSITIONS AND METHODS FOR MITIGATING INADEQUATE NUTRITIONAL INTAKE OF AN ALTERNATE DAY FASTING REGIMEN

(71) Applicant: SOCIETE DES PRODUITS NESTLE S.A., Vevey (CH)

(72) Inventors: PALOMA ELORTEGUI PASCUAL, Epalinges (CH); ALISON L. ELDRIDGE, Pully (CH); ERIC ANTOINE SCUCCIMARRA, Lausanne (CH)

(21) Appl. No.: 18/569,268

(22) PCT Filed: Jun. 15, 2022
(86) PCT No.: PCT/EP2022/066250

§ 371 Date: Dec. 12, 2023

(30) Foreign Application Priority Data
Jun. 15, 2021 (EP) 21179456.5

(51) Int. Cl. G16H 20/60; G16H 10/60; G16H 50/30

(52) U.S. Cl. CPC G16H 20/60; G16H 10/60; G16H 50/30

(57) ABSTRACT

The present invention relates to novel nutritional compositions and methods to mitigate inadequate nutrient intakes of intermittent fasting (IF) diets, in particular alternate day fasting regimens (ADF). It also covers an Artificial Intelligence (AI)-based system for determination, quantification and mitigation of nutritional risks of said ADF diets in adults.

### FIELD OF THE INVENTION

The present invention relates to novel nutritional compositions and methods to mitigate inadequate nutrient intakes of intermittent fasting (IF) diets, in particular alternate day fasting regimens (ADF). It also covers an Artificial Intelligence (AI)-based system for determination, quantification and mitigation of nutritional risks of said ADF diets in adults.

### BACKGROUND OF THE INVENTION

Intermittent fasting (IF) is a generic term to describe dietary patterns that include a period where little or no energy intake is consumed, alternating with a feeding—sometimes referred to as feasting—period. One of the most popular styles of IF is Alternate Day Fasting (ADF) where 24-hour fasting periods alternate with 24-hours of ad libitum intake. There are three common versions of ADF:
Strict Alternate Day Fasting where people alternate one day of eating with one day of fasting. In this regimen, no calories are consumed on the fasting day, but non-caloric beverages (e.g., water, infusions, tea, coffee) may be consumed.
Modified Alternate Day Fasting where people alternate one day of eating with one day of a modified fast, consuming foods and beverages with a limited number of calories (e.g., ≤500 kcal) on the fasting day.
5:2 regimen where people fast two out of every seven days. Note: The 5:2 regimen also includes modifications where on two out of seven days, a limited number of calories can be consumed.

ADF is generally used as a weight loss regimen, but IF has also been purported to have many other health benefits including glycemic control, cardiovascular health, reduced inflammation, improved cognition and healthy (de Cabo R, Mattson MP. Effects of intermittent fasting on health, aging and disease, New Engl J Med. 2019; 381:2541-51. DOI: 10.1056/NEJMra1905136). Efficacy of IF on health parameters has been reported in animal and human studies, but results vary widely in the literature, most likely due to the variability in fasting regimens and study designs.

A person following an ADF diet typically will not be aware of possible nutrient inadequacies stemming from their diet or the amounts of those inadequacies. These two points cannot be easily answered by existing clinical trials because real-life eating patterns may differ from the dietary choices given under controlled conditions and constant supervision in a clinical setting. In addition, the groups studied may not be representative of those self-selecting an ADF diet. Therefore, the nutrients identified as at risk in clinical settings can differ from those in real-life.

Furthermore, constant daily reporting and analysis of an individual's diet is not practicable because daily recording is tedious, people don't accurately report what they have eaten and may forget to report individual items and eating occasions. Most people do not have access to nutritional databases to assess the composition of the foods and beverages they consume, and they lack the expertise needed to identify nutrient inadequacies.

Accordingly, there is a need to provide solutions in the form of nutritional compositions and recommendations to prevent nutrient inadequacies in individuals following an Alternate Day Fasting regimen.

### SUMMARY OF THE INVENTION

The present invention addresses the inadequate nutrient intakes in the state of the art by providing new nutritional recommendations and innovative methods for personalized nutrient, dietary and lifestyle recommendations for individuals who are following or planning to follow an Alternate Day Fasting regimen (e.g., strict ADF, Modified ADF or 5:2).

In a first aspect, the present invention provides a computer implemented method or AI-based system to identify and/or quantify individual nutritional inadequacies in individuals following or planning to follow an Alternate Day Fasting regimen, comprising:
(i) Identifying the individual's nutritional needs and/or dietary rules,
(ii) Comparing the individual's nutritional needs and/or dietary rules with the nutrients provided by the ADF diet, and
(iii) Performing analyses to identify and quantify the potential nutritional inadequacies.

In an embodiment, the individual's nutritional needs are based on one or more of the following personal characteristics: age, gender, height, weight, physical activity, lifestyle and medical condition.

In another embodiment the dietary rules are based on individual's specific dietary restrictions (e.g., gluten-free, lactose-free, etc.) or preferences (e.g., Mediterranean Diet, Flexitarian, Vegetarian (with and without dairy and eggs), Vegan, etc.).

In an embodiment the ADF diet is a simulated ADF diet.

In another aspect, the present invention addresses the specific condition of dietary inadequate intakes of an Alternate Day Fasting regimen, by providing novel, consolidated dietary recommendations. More particularly, the present invention provides a computer implemented method or AI-based system to mitigate nutrient inadequacies in an individual following or planning to follow an Alternate Day Fasting (ADF) regimen, said method comprising:
i) Identifying nutritional inadequacies in the context of a defined ADF diet,
ii) Providing specific dietary recommendations and/or nutritional solutions to mitigate the identified nutritional inadequacies in said individual, and
iii) Optionally providing recommendations on lifestyle.

In an embodiment, the method to identify nutritional inadequacies is as described above so that in another aspect, the present invention provides a method of mitigating nutrient inadequacies in an individual following or planning to follow an Alternate Day Fasting regimen, said method comprising:
i) Identifying the individual's nutritional needs and/or dietary rules,
ii) Comparing the individual's nutritional needs and/or dietary rules with the nutrients provided by an ADF diet,
iii) Performing analyses to identify and quantify potential nutritional inadequacies,
iv) Providing specific dietary recommendations and/or nutritional solutions to mitigate the identified nutritional inadequacies in said individual, and
v) Optionally providing recommendations on lifestyle.

In an embodiment, the nutritional solutions include foods, beverages and/or dietary supplements thus recommended in the context of defined ADF dietary pattern, lifestyle and dietary rules.

An advantage of the present invention is to aid dietary decision-making before the user takes the decision to change its habitual diets and guide on needed dietary habit changes when following an ADF diet. It provides an estimation of prospective nutritional status by simulating the chosen diet and analyzing possible nutritional gaps in advance.

Another advantage of the present invention is to help individuals following or planning to follow an ADF diet to establish a personalized nutritional risk mitigation plan. In particular, it can provide awareness to diet followers on nutritional risks of inadequate intake when following such a diet via AI-based and nutrition science models.

A further advantage of the present invention is to prompt users to consume foods, beverages and/or dietary supplements to fulfill nutrient requirements specific to their ADF regimen.

### BRIEF DESCRIPTION OF FIGURES

FIG. 1. Calcium intake ranges comparing simulated baseline intakes with Strict ADF, Modified ADF and 5:2 for males. The boxes show the 25th percentile (lower bound) and 75th percentile (upper bound) of intakes for each diet. The bar in the middle of each box is the Median intake. All ADF regimens have median intakes lower than the median at baseline. Median intakes at baseline exceed the RDA for calcium. Median intakes of calcium for 5:2 also exceeds the RDA, but median intakes for both Strict ADF and Modified ADF and below the RDA.

FIG. 2. Calcium intake ranges comparing simulated baseline intake with Strict ADF, Modified ADF and 5:2 for females. The boxes show the 25th percentile (lower bound) and 75th percentile (upper bound) of intakes for each diet. The bar in the middle of each box is the Median intake. All ADF regimens have median intakes lower than the median at baseline. Median intakes at baseline are below the RDA, but above the EAR. Median intakes of calcium for all ADF diets are below the EAR.

FIG. 3. Fiber intake ranges comparing simulated baseline intakes with Strict ADF, Modified ADF and 5:2 for males. The boxes show the 25th percentile (lower bound) and 75th percentile (upper bound) of intakes for each diet. The bar in the middle of each box is the Median intake. Fiber intakes at baseline and for all ADF diets are below the Al. Median fiber intakes are lowest for Strict ADF, followed Modified ADF

FIG. 4. Fiber intake ranges comparing simulated baseline intakes with Strict ADF, Modified ADF and 5:2 for females. The boxes show the 25th percentile (lower bound) and 75th percentile (upper bound) of intakes for each diet. The bar in the middle of each box is the Median intake. The Median intakes of fiber for females at baseline is above the AI. Median intakes for fiber on all ADF diets are below the AI, with lowest intakes in Strict ADF.

### DETAILED DESCRIPTION OF THE INVENTION

### Definitions

Prior to discussing the present invention in further details, the following terms and conventions will first be defined.

Types of Alternate Day Fasting (ADF) include the following:
Strict Alternate Day Fasting where people alternate one day of eating with one day of fasting. In this regimen, no calories are consumed on the fasting day, but non-caloric beverages (e.g., water, infusions, tea, coffee) may be consumed.
Modified Alternate Day Fasting where people alternate one day of eating with one day of a modified fast, consuming foods and beverages with a limited number of calories (e.g. ≤500 kcal) on the fasting day.
5:2 regimen where people fast two out of every seven days. Note: The 5:2 regimen also includes modifications where on two out of seven days, a limited number of calories can be consumed.

Within the context of the present invention, “nutrients” are substances needed for health, growth, development and functioning of an organism, including:
Macronutrients (e.g., protein, carbohydrates, fats) and their components (e.g., amino acids, sugars, starches, fatty acids, etc.),
Micronutrients (e.g., vitamins, minerals),
Other food components (fiber, cholesterol, bioactive phytochemicals, alcohol, etc.),
Water contained in foods and beverages.

The term “composition” can mean a food, beverage, dietary supplement, complete nutrition or oral nutritional supplement (ONS) or medical food composition, or mixture thereof.

Within the context of the present invention, the terms “food,” “food product” and “food composition” mean a product or composition that is intended for ingestion by an individual such as a human and provides nutritional support to an organism, including those that provide energy, nutrients, and water. The compositions of the present disclosure, including the many embodiments described herein, can comprise, consist of, or consist essentially of one or more of the nutrients listed above, as well as any additional or optional ingredients or components safe for human consumption and otherwise useful in a diet.

Within the context of the present invention, the term “beverage,” “beverage product” and “beverage composition” mean a potable liquid product or composition for ingestion by an individual such as a human and provides water and may also include one or more nutrients and other ingredients safe for human consumption to the individual. The compositions of the present disclosure, including the many embodiments described herein, can comprise, consist of, or consist essentially of one or more of the nutrients listed above, as well as any additional or optional ingredients, components safe for human consumption and otherwise useful in a diet.

Within the context of the present invention, “dietary supplements” are products taken by mouth that contain one or more dietary ingredient, such as vitamins, minerals, amino acids, fatty acids, fibers and/or herbs and other botanical ingredients used to supplement the diet. Dietary supplements come in many forms and may be available as tablets, capsules, powders, liquids, and formulated into specific foods, such as “energy” bars.

As used herein, “complete nutrition” contains sufficient types and levels of macronutrients (protein, fats and carbohydrates), micronutrients, and other food components to be sufficient to be a sole source of nutrition for the subject to which the composition is administered. Individuals can receive 100% of their nutritional requirements from such complete nutritional compositions.

Within the context of the present invention, the term “Dietary Reference Intakes (DRIs)” indicates a set of reference values used to plan and assess the nutrient intakes of healthy people. The DRIs were established by the United States and Canadian governments, and published by the National Academies of Sciences, Engineering, and Medicine (NASEM; formerly called Institute of Medicine (IOM); https://www.nal.usda.gov/fnic/dri-nutrient-reports). Within the context of the present invention, the terms used to describe the DRIs (Institute of Medicine (US) Food and Nutrition Board. Dietary Reference Intakes: A Risk Assessment Model for Establishing Upper Intake Levels for Nutrients. Washington DC, USA: National Academies Press; 1998. What are Dietary Reference Intakes?) are as follows:
Recommended Dietary Allowance (RDA) indicates the average daily intake level of a nutrient considered sufficient to meet the requirements of 97.5% of healthy individuals. RDAs vary by gender, age, and whether a woman is pregnant or lactating. RDAs are calculated based on the Estimated Average Requirements (EAR) and is usually approximately 20% higher than the EAR.
Adequate Intake (AI) for a nutrient is the amount estimated to meet or exceed the amount needed to maintain adequate nutrition for most people in a specific age or gender group. An AI is set instead of an RDA when there is insufficient evidence to calculate an EAR.
Tolerable Upper Intake Level (UL) is the highest amount of daily nutrient intake considered to pose no risk of adverse health effects for most people. Consuming more than the UL increases the risk of adverse effects from over-consumption.
Estimated Average Requirement (EAR) for nutrients are calculated to meet the requirements of 50% of the people in a specific age and gender group.

The EAR is required to establish an RDA. If the standard deviation (SD) of the EAR is available and the requirement for the nutrient is symmetrically distributed, the RDA is set at two SDs above the EAR:

$$RDA = EAR + 2 SD ( EAR )$$

If data about variability in requirements are insufficient to calculate a SD, a coefficient of variation (CV) for the EAR of 10 percent is assumed, unless available data indicate a greater variation in requirements. If 10 percent is assumed to be the CV, then twice that amount when added to the EAR is defined as equal to the RDA. The resulting equation for the RDA is:

RDA=1.2 (EAR)

Different national and regional authorities have different dietary reference values. For example, The European Food Safety Authority (EFSA) refers to the collective set of information as Dietary Reference Values (DRV), with Population Reference Intake (PRI) instead of RDA, and Average Requirement instead of EAR. Al and UL are defined the same as in United States, but values may differ (EFSA Panel on Dietetic Products, Nutrition, and Allergies (NDA). Scientific Opinion on Principles for Deriving and Applying Dietary Reference Values. EFSA J. 2020;8(3):1458. https://doi.org/10.2903/j.efsa.2010.1458). These standards and values are also used in the context of this invention.

Within the context of the present invention, the terms “nutrient inadequacy” or “dietary inadequacy” indicates that the total daily dietary intake of a nutrient in a certain individual is below the Estimated Average Requirements (EARs) for said individual and/or below well-established nutritional requirements.

Within the context of the present invention, the expression “prevent nutrient inadequacy” should be understood to include prevention of inadequacies of the nutrient or nutrients, as well as reduction of the risk of nutrient inadequacies in the individual following an ADF diet.

Within the context of the present invention, numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 10 (including 1 and 10), from 2 to 8, from 3 to 7, from 1 to 9, from 3.6 to 4.6, from 3.5 to 9.9, and so forth. All references to singular characteristics or limitations of the present invention shall include the corresponding plural characteristic or limitation, and vice versa, unless otherwise specified or clearly implied to the contrary by the context in which the reference, is made.

Within the context of the present invention, the term “and/or” used in the context of the “X and/or Y” should be interpreted as “X”, or “Y”, or “X and Y”.

Unless defined otherwise, all technical and scientific terms used herein have the same meaning as commonly understood by one of ordinary skill in the art.

### Embodiments

The present invention provides a computer implemented method or AI-based system for providing nutritional recommendations to mitigate nutrient inadequacies in subjects following or planning to follow an Alternate Day Fasting diet (including Strict ADF, Modified ADF or 5:2).

In a first aspect, the present invention provides a computer implemented method or AI-based system to identify and/or quantify nutritional inadequacies in an individual following or planning to follow an Alternate Day Fasting regimen, comprising the steps of:
(i) Identifying the individual's nutritional needs and/or dietary rules,
(ii) Comparing the individual's nutritional needs and/or dietary rules with the nutrients provided by an ADF diet, and
(iii) Performing analyses to identify and quantify the potential nutritional inadequacies.

In an embodiment, the Alternate Day Fasting regimen may be one of Strict ADF, Modified ADF or 5:2 protocols.

In another embodiment, the individual's nutritional needs are based on one or more of the following individual's characteristics: age, gender, height, weight, physical activity, lifestyle and medical condition. In a preferred embodiment, the nutritional needs are based at least on gender, age, weight height and physical activity.

In another embodiment the dietary rules are based on individual's specific dietary restrictions (e.g., gluten-free, lactose-free, etc.) or preferences (e.g., Mediterranean Diet, Flexitarian, Vegetarian (with and without dairy and eggs), Vegan, etc.).

In another embodiment the ADF diet is a simulated ADF diet.

### General Diet Simulator Methodology

In a preferred embodiment, to simulate the different ADF diets and identify potential nutrient inadequacies associated with each diet, we conduct the following steps:
1. Simulate N=20000 possible days of dietary intakes, 10000 each for males and females, for each ADF protocol, where days of dietary intakes were defined as a set of foods and beverages and their amounts assigned for different eating occasions (e.g., breakfast, lunch, dinner and 1 snack) per day, where each simulation follows certain requirements:
   Meet the person's energy needs based on age, gender, height, weight, physical activity level,
   Follow ADF rules, defined by each diet protocol type (e.g., Strict ADF, Modified ADF and 5:2), and
   Provide realistic amounts of foods and beverages and their combinations to generate daily nutrient totals.
2. Compare the person's individual nutritional needs (depending on age, gender, height, weight, physical activity, etc.) with the nutrients provided by the simulated ADF diet.
3. Perform analysis (e.g. statistical analysis) to identify and quantify potential nutritional inadequacies.

Ultimately, said method is used to recommend foods, beverages and dietary supplements that best close the identified nutritional inadequacies/gaps in the context of defined Alternate Day Fasting dietary patterns.

By making certain assumptions, the system or method can provide a reasonable estimate of the nutritional inadequacies/gaps and thus provide recommendations on how to close those gaps without requiring undue effort or input from the user.

It is important to note that specific nutritional inadequacies/gaps will depend on dietary habits and cultural characteristics and will likely differ in different populations around the world. The system or method described here will be the same, but the nutritional inadequacies identified may differ. The example provided hereafter, relies on food group level diet recommendations as specified by the USDA Healthy Eating Patterns which provide target amounts of food groups to be consumed on a daily and weekly basis. The simulation tool can easily be adapted to different food-based dietary guidelines if applied in other geographies.

In another aspect, the present invention aims to provide novel, consolidated dietary recommendations to address the specific condition of dietary inadequate intake of an intermittent fasting diet, which combine:
Specific dietary intake amounts by gender, age, and intermittent fasting protocol followed or to be followed,
Specific dietary recommendations to be consumed daily by gender and intermittent fasting protocol followed or to be followed, and
Specific recommendations on lifestyle (e.g., physical activity).

More particularly, the present invention provides a computer implemented method or AI-based system to mitigate nutrient inadequacies in an individual following or planning to follow an Alternate Day Fasting regimen, said method comprising the steps of:
i) Identifying nutritional inadequacies in the context of defined ADF dietary pattern, lifestyle and/or dietary rules,
ii) Providing specific dietary recommendations and/or nutritional solutions to mitigate the identified nutritional inadequacies in said individual, and
iii) Optionally providing recommendations on lifestyle.

In an embodiment, the method to identify and/or quantify the nutritional inadequacies is as described above. Additional criteria could be specified such as foods, food groups, and/or specific nutrients which should be excluded, minimized or maximized.

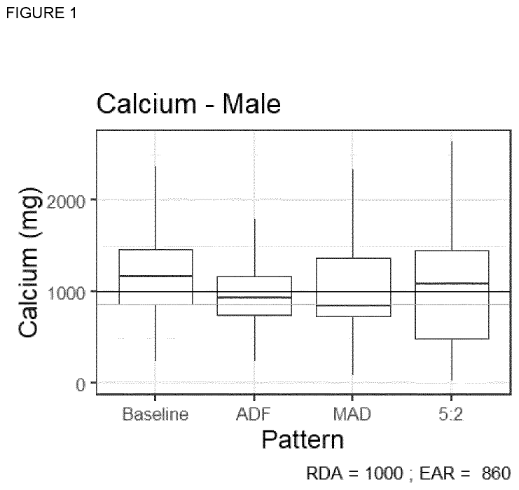

In a preferred embodiment, in order to construct dietary recommendations (as described in more details in the example section), we consider the upper and lower bounds of the intake recommendations to identify nutrient targets, without exceeding the UL (see FIGS. 1-4 for examples for calcium and folate). The “Amount of Inadequacy” is used to generate the dietary recommendations. First, we take the difference between the RDA, or the AI if no RDA is available (or any relevant guideline), and the 2.5% intake percentile as the upper and the difference with the 97.5% intake percentile as the lower. It is understood that any other percentile can be used. All recommendations are preferably based on an energy level for the diet of 2000 kcal. Alternatively, the recommendation may be based on a different energy level if desired. The upper end of the recommendation is capped to ensure that the recommendation does not exceed the UL.

In a further embodiment, a computerized Diet Simulator may be used. By making certain assumptions, the Diet Simulator can provide a reasonable estimate of the nutritional inadequacies/gaps and thus provide recommendations on how to close those gaps from nutrients, foods, beverages, and/or dietary supplements, and their combinations, without requiring undue effort or input from the user.

In an embodiment, the dietary recommendations comprise recommendations related to one or more of the following:
Nutrients, and/or
Foods and beverages, and/or
Dietary supplements, and/or
Combinations of foods and beverages as menu recommendations and/or recipe recommendations.

In an embodiment, the recommendations for nutrients, foods, beverages, and dietary supplements are selected to best close the identified nutritional inadequacies/gaps in the context of the defined ADF regimen. Said recommendations are based on the individual's characteristics, diet rules and ADF dietary pattern.

The system according to the present invention is a Diet Simulator, comprises at least one of the following components:
A food composition database,
A recipe database,
A database of meals,
A nutrient scoring module, and
An optimization module.

The compositions disclosed herein are intended to be consumed orally. As such, non-limiting examples of the form of the composition include natural foods, processed foods (including but not limited to milling, grinding, baking, drying, fermenting, canning, freezing, pasteurizing, extruding, cooking, and other processing methods to make raw food ingredients palatable and ready-to-eat), creamers, beverages such as coffee based beverages, natural juices, concentrates and extracts.

In several embodiments, the present invention may include menu suggestions or recipes that incorporate foods, beverages and/or dietary supplements to alleviate the nutritional inadequacies or excesses caused by the ADF regimen. Depending on the ADF regimen, these foods, beverages, and/or dietary supplements as well as the menus and recipes that use them can be recommended for consumption on a daily basis, or on eating days for the duration of the individual's adherence to the ADF regimen.

In several embodiments, the present invention may also package foods, beverages and/or dietary supplements into an ADF “kit” of parts comprising meal recommendations, recipes, or menus for individuals on an ADF diet, wherein said kit includes a composition according to the invention.

### EXAMPLE

Risk of inadequate nutrient intakes of intermittent fasting protocols with simulated diets

In order to estimate the potential risk of nutrients inadequacies for ADF diets, without the use of randomized controlled trials (RCT) or traditional dietary assessment methods (e.g., 24-hours food recall, food frequency questionnaire, food records), we simulated approximately 20,000 days of food intake in silico for a variety of individuals adhering to an IF diet with a digital tool.

The digital simulation tool simulates multiple days of food intake by finding the optimal combination of available meals to maximize nutritional intakes. In order to mimic real-world food intake as closely as possible, we used actual meals consumed by people as reported in the National Health and Nutrition Examination Survey (NHANES), a survey performed by the US Center for Disease Control and Prevention (CDC). Datasets were downloaded from https://wwwn.cdc.gov/nchs/nhanes/default.aspx. The NHANES cycles 2013-2014, 2015-2016, 2017-2018 were used, more specifically, the files “Dietary Interview-Individual Foods, First Day,” and “Demographic Variables and Sample Weights.” This allows for the simulated diets to consist of meals actually consumed by the US population.

It is important to note that specific nutritional inadequacies will depend on dietary habits and cultural characteristics and will likely differ in different populations around the world. The system or method described here will be the same, but the nutritional inadequacies identified may differ.

Within the simulation tool, the user specifies the characteristics for a set of individuals based on sex, age, height, weight and physical activity level in order to calculate the estimated energy requirement (EER) per day. The simulation tool then uses integer programming techniques to create in silico menu plans which optimize the nutritional content of the overall diet. In this Example, the simulation tools rely on food group level diet recommendations as specified by the USDA Healthy Eating Patterns which provide target amounts of food groups to be consumed on a daily and weekly basis. (The simulation tool can be adapted to different food-based dietary guidelines if applied in other geographies.)

Additional nutrient level constraints are set as upper limits on nutrients such as sodium, added sugars, and saturated fats. The constraints are adjusted based on the individual's EER to obtain desired ranges of food groups and nutrients to be consumed per day and per week. An objective function is created as a scalar weighted linear combination of the differences between the actual amount of each individual food group and nutrient and the optimal range. The diet is created by selecting the combination of meals which meet the energy requirements while attempting to keep the consumption of food groups and nutrients within the optimal range.

### Baseline Diet Simulation

To simulate the dietary intakes of different ADF regimens in a manner such that the results for the various protocols (Strict ADF, Modified ADF and 5:2) are comparable, we simulated approximately 20,000 days of “baseline” diet by maximizing the Healthy Eating Patterns as described above with no other rules applied. This “baseline” diet is considered to be a healthy and balanced diet (Table 1). In this Example, the baselines were simulated for 1,400 individuals of each gender, ranging in age from 18 to 70, of varying heights and weights and sedentary physical activity level, for 7 days each. While the use of meals extracted from NHANES forces the simulated diets to use meals reportedly consumed by actual people, the optimization of the balance of food groups produces diets with a bias towards healthier diets than what people are likely to actually eat. As such we considered these to be “ideal” diets rather than realistic diets, which will tend to have higher nutrient intakes than occur in the real world.

TABLE 1
Percent of individuals with inadequate intakes
at baseline (from diet simulation models).

| | Males | | | Females | | |
|---|---|---|---|---|---|---|
| | Mean Intake | | Percent | Mean Intake | | Percent |
| Nutrient | Baseline | | Inadequate | Baseline | | Inadequate |
| Total fat | 47.1 | g | 0 | 41.7 | g | 0 |
| Fiber | 28.9 | g | 77 | 27.4 | g | 17 |
| Calcium | 1240.1 | mg | 9 | 1090.7 | mg | 41 |
| Copper | 1.7 | μg | 0 | 1.5 | μg | 0 |
| Food folate | 387.4 | μg | 12 | 376.0 | μg | 30 |
| Iron | 14.3 | mg | 0 | 12.2 | mg | 3 |
| Magnesium | 402.3 | mg | 2 | 370.8 | mg | 4 |
| Niacin | 20.9 | mg | 0 | 17.4 | mg | 7 |
| Potassium | 5121.9 | mg | 0 | 4620.0 | mg | 0 |
| Riboflavin | 2.3 | mg | 0 | 2.0 | mg | 0 |
| Selenium | 93.2 | μg | 0 | 77.4 | μg | 9 |
| Sodium | 2256.2 | mg | 1 | 1931.4 | mg | 24 |
| Vitamin A | 1076.1 | μg | 9 | 875.3 | μg | 23 |
| Vitamin B12 | 4.2 | μg | 2 | 3.3 | μg | 17 |
| Vitamin B6 | 3.0 | mg | 0 | 2.6 | mg | 1 |
| Vitamin C | 496.4 | mg | 0 | 479.5 | mg | 0 |
| Vitamin D | 7.5 | μg | 59 | 5.6 | μg | 86 |
| Vitamin E | 8.2 | mg | 94 | 8.0 | mg | 93 |
| Vitamin K | 113.7 | μg | 80 | 122.2 | μg | 52 |
| Zinc | 10.6 | mg | 18 | 9.0 | mg | 25 |

It is noted that some nutrients are low at baseline, meaning they are not at risk particularly because of IF diets, but in general in US diet/baseline.

### ADF Protocol Diet Simulation

The ADF protocols were created from the baseline diets by removing meals as per the rules of each ADF protocol:
Strict Alternate Day Fasting: every other day no food at all, with energy increased by 25% to mimic the increased energy intake typically consumed on eating days,
Modified Alternate Day Fasting: every other day all meals but lunch are removed, with lunch meal restricted to 400-600 kcal per day, and
5:2 on days 4 and 7 of every week all meals but breakfast are removed (to leave about 25% of daily calories, equivalent to about 500 kcal for someone consuming 2000 kcal/day).

By comparing the baseline inadequacies to the different ADF protocols, we can identify how nutrient intakes tend to change by following the various protocols (Table 2). This method of simulation, where a baseline diet is modified to comply with the rules of the IF protocol, forces the results to be comparable, allowing us to identify the relative risks of nutrient inadequacy for the various protocols while controlling for randomness inherent in the simulation process and for nutrient inadequacies which may result from the simulation process rather than being inherent in the ADF regimen.

TABLE 2
Percent of individuals with inadequate intakes when rules for
each ADF regimen were applied (from diet simulation models).

| | Percent of Inadequacy, | | | Percent of Inadequacy, | | |
|---|---|---|---|---|---|---|
| | Males | | | Females | | |
| | Strict | Modified | | Strict | Modified | |
| Nutrient | ADF | ADF | 5:2 | ADF | ADF | 5:2 |
| Total fat | 0.0 | 0.0 | 0.0 | 5.8 | 2.3 | 2.5 |
| Fiber | 98.9 | 97.0 | 97.5 | 85.4 | 76.6 | 71.0 |
| Calcium | 33.6 | 28.8 | 24.4 | 62.1 | 54.8 | 54.8 |
| Copper | 0.0 | 0.0 | 0.0 | 4.3 | 4.0 | 3.0 |
| Food Folate | 77.9 | 50.4 | 60.4 | 74.9 | 60.8 | 59.5 |
| Iron | 1.1 | 0.6 | 0.9 | 23.9 | 17.0 | 15.3 |
| Magnesium | 83.6 | 76.8 | 63.4 | 46.3 | 36.0 | 32.4 |
| Niacin | 8.8 | 3.6 | 5.0 | 34.4 | 21.3 | 21.1 |
| Potassium | 3.1 | 3.3 | 0.9 | 2.2 | 1.6 | 0.8 |
| Riboflavin | 2.9 | 3.3 | 2.0 | 8.9 | 7.4 | 6.8 |
| Selenium | 2.6 | 1.7 | 1.1 | 34.6 | 24.0 | 23.2 |
| Sodium | 9.0 | 5.4 | 6.1 | 59.9 | 45.9 | 45.1 |
| Vitamin A | 17.6 | 17.1 | 13.1 | 36.0 | 31.7 | 31.8 |
| Vitamin B12 | 10.0 | 7.6 | 6.6 | 43.4 | 37.6 | 40.4 |
| Vitamin B6 | 0.5 | 0.6 | 0.1 | 10.1 | 8.9 | 7.2 |
| Vitamin C | 0.0 | 0.0 | 0.0 | 0.0 | 0.0 | 0.0 |
| Vitamin D | 90.4 | 89.0 | 85.0 | 98.7 | 95.8 | 94.9 |
| Vitamin E | 98.9 | 99.1 | 98.1 | 98.5 | 98.0 | 97.9 |
| Vitamin K | 92.7 | 92.1 | 92.1 | 70.5 | 67.5 | 63.9 |
| Zinc | 67.4 | 61.9 | 57.0 | 5.9 | 45.5 | 45.8 |

The diet for each simulated individual is analyzed by comparing the mean intakes of each nutrient to the DRI for that individual, based on their age and gender. As nutrient intakes tend to not be normally distributed, bootstrap confidence intervals are created for the mean which are compared to the appropriate DRI. In order to pool results for individuals with varying DRIs, the result for each individual is calculated as a ratio of their mean intake to their DRI. The overall inadequacies/gaps between simulated intake and DRI are created by taking the mean of these ratios. The risks of inadequacies are created by taking the percentage of simulated individuals with inadequate intake for each nutrient.

The results of the analyses and the resulting recommendations for each group are below. These tables show the complete results of the analysis.

### Risk of Inadequate Intakes by ADF Regimen

Tables 3-8 show the risk for inadequacies identified for males and females for each ADF regimen. Most nutrients have an EAR, but when there were not sufficient data to assign an EAR, an AI was assigned instead (Institute of Medicine, US). In the Tables below, “Mean Inadequacy” is the amount below EAR or AI. The “Percent Inadequate” indicates the percent of individuals below the EAR or AI. If a large portion of the modelled diets produce inadequacies, then those nutrients are considered to be “At Risk” for inadequacies. Note that the interpretation is somewhat different for the AI; with a percent above an AI, we can generally assume adequacy, but a percent below does not necessarily imply inadequacy. A negative Mean Inadequacy indicates that the intakes tend to be adequate.

TABLE 3
Nutritional Analysis of Strict ADF for Males.

| | | | EAR/ | | | |
|---|---|---|---|---|---|---|
| | DRI | | AI | Mean | Percent | Assess- |
| Nutrient | Type | Units | Value | Inadequacy | Inadequate | ment |
| Total fat | EAR | g | 20 | −17.5 | 0.0 | |
| Fiber | AI | g | 34.8 | 13.1 | 98.9 | At Risk |
| Calcium | EAR | mg | 860 | −132 | 33.6 | At Risk |
| Copper | EAR | mcg | 0.7 | −0.6 | 0.0 | |
| Food folate | EAR | mcg | 320 | 35.8 | 77.9 | At Risk |
| Iron | EAR | mg | 6 | −5.7 | 1.1 | |
| Magnesium | EAR | mg | 346 | 36.1 | 83.6 | At Risk |
| Niacin | EAR | mg | 12 | −5.5 | 8.8 | |
| Potassium | AI | mg | 3000 | −1010 | 3.1 | |
| Riboflavin | EAR | mg | 1.1 | −0.78 | 2.9 | |
| Selenium | EAR | mcg | 45 | −32.8 | 2.6 | |
| Sodium | AI | mg | 1500 | −344 | 9.0 | |
| Vitamin A | EAR | mcg | 625 | −283 | 17.6 | |
| Vitamin B12 | EAR | mcg | 2. | −1.7 | 10.0 | |
| Vitamin B6 | EAR | mg | 1.22 | −1.25 | 0.5 | |
| Vitamin C | EAR | mg | 75 | −290.7 | 0.0 | |
| Vitamin D | EAR | mcg | 10 | 3.3 | 90.4 | At Risk |
| Vitamin E | EAR | mg | 12 | 6.0 | 98.9 | At Risk |
| Vitamin K | AI | mcg | 120 | 44.5 | 92.7 | At Risk |
| Zinc | EAR | mg | 9.4 | 0.7 | 67.4 | At Risk |

TABLE 4
Nutritional Analysis of Strict ADF for Females.

| | | | EAR/ | | | |
|---|---|---|---|---|---|---|
| | DRI | | AI | Mean | Percent | Assess- |
| Nutrient | Type | Units | Value | Inadequacy | Inadequate | ment |
| Total fat | EAR | g | 18.4 | −11.3 | 5.8 | |
| Fiber | AI | g | 23.4 | 3.8 | 85.4 | At Risk |
| Calcium | EAR | mg | 880 | 101 | 62.1 | At Risk |
| Copper | EAR | mcg | 0.7 | −0.4 | 4.3 | |
| Food folate | EAR | mcg | 320 | 50.8 | 74.9 | At Risk |
| Iron | EAR | mg | 6.9 | −1.8 | 23.9 | |
| Magnesium | EAR | mg | 263 | −2.2 | 46.3 | At Risk |
| Niacin | EAR | mg | 11 | −1.4 | 34.4 | At Risk |
| Potassium | AI | mg | 2300 | −1003 | 2.2 | At Risk |
| Riboflavin | EAR | mg | 0.9 | −0.50 | 8.9 | |
| Selenium | EAR | mcg | 45 | −10.3 | 34.6 | At Risk |
| Sodium | AI | mg | 1500 | 121 | 59.9 | |
| Vitamin A | EAR | mcg | 500 | −127 | 36.0 | At Risk |
| Vitamin B12 | EAR | mcg | 2 | −0.3 | 43.4 | At Risk |
| Vitamin B6 | EAR | mg | 1.18 | −0.67 | 10.1 | |
| Vitamin C | EAR | mg | 60 | −282.8 | 0.0 | |
| Vitamin D | EAR | mcg | 10 | 6.0 | 98.7 | At Risk |
| Vitamin E | EAR | mg | 12 | 6.3 | 98.5 | At Risk |
| Vitamin K | AI | mcg | 90 | 2.3 | 70.5 | At Risk |
| Zinc | EAR | mg | 6.8 | 0.4 | 5.9 | At Risk |

TABLE 5
Nutritional Analysis of Modified ADF for Males.

| | | | EAR/ | | | |
|---|---|---|---|---|---|---|
| | DRI | | AI | Mean | Percent | Assess- |
| Nutrient | Type | Units | Value | Inadequacy | Inadequate | ment |
| Total fat | EAR | g | 20 | −20.6 | 0.0 | |
| Fiber | AI | g | 34.8 | 12.5 | 97.0 | At Risk |
| Calcium | EAR | mg | 860 | −166 | 28.8 | At Risk |
| Copper | EAR | mcg | 0.7 | −0.6 | 0.0 | |
| Food folate | EAR | mcg | 320 | 16.2 | 50.4 | At Risk |
| Iron | EAR | mg | 6 | −5.9 | 0.6 | |
| Magnesium | EAR | mg | 346 | 25.0 | 76.8 | At Risk |
| Niacin | EAR | mg | 12 | −5.7 | 3.6 | |
| Potassium | AI | mg | 3000 | −1126 | 3.3 | |
| Riboflavin | EAR | mg | 1.1 | −0.8 | 3.3 | |
| Selenium | EAR | mcg | 45 | −40.3 | 1.7 | |
| Sodium | AI | mg | 1500 | −542 | 5.4 | |
| Vitamin A | EAR | mcg | 625 | −413 | 17.1 | |
| Vitamin B12 | EAR | mcg | 2. | −1.6 | 7.6 | |
| Vitamin B6 | EAR | mg | 1.22 | −1.3 | 0.6 | |
| Vitamin C | EAR | mg | 75 | −305.2 | 0.0 | |
| Vitamin D | EAR | mcg | 10 | 3.0 | 89.0 | At Risk |
| Vitamin E | EAR | mg | 12 | 5.5 | 99.1 | At Risk |
| Vitamin K | AI | mcg | 120 | 40.7 | 92.1 | At Risk |
| Zinc | EAR | mg | 9.4 | 0.4 | 61.9 | At Risk |

TABLE 6
Nutritional Analysis of Modified ADF for Females.

| | | | EAR/ | | | |
|---|---|---|---|---|---|---|
| | DRI | | AI | Mean | Percent | Assess- |
| Nutrient | Type | Units | Value | Inadequacy | Inadequate | ment |
| Total fat | EAR | g | 18.4 | −15.3 | 2.3 | |
| Fiber | AI | g | 23.4 | 2.7 | 76.6 | At Risk |
| Calcium | EAR | mg | 880 | 32 | 54.8 | At Risk |
| Copper | EAR | mcg | 0.7 | −0.5 | 4.0 | |
| Food folate | EAR | mcg | 320 | 26.0 | 60.8 | At Risk |
| Iron | EAR | mg | 6.9 | −2.4 | 17.0 | |
| Magnesium | EAR | mg | 263 | −22.9 | 36.0 | At Risk |
| Niacin | EAR | mg | 11 | −2.5 | 21.3 | At Risk |
| Potassium | AI | mg | 2300 | −1246 | 1.6 | At Risk |
| Riboflavin | EAR | mg | 0.9 | −0.6 | 7.4 | |
| Selenium | EAR | mcg | 45 | −17.6 | 24.0 | At Risk |
| Sodium | AI | mg | 1500 | −72.1 | 45.9 | |
| Vitamin A | EAR | mcg | 500 | −216 | 31.7 | At Risk |
| Vitamin B12 | EAR | mcg | 2 | −0.5 | 37.6 | At Risk |
| Vitamin B6 | EAR | mg | 1.18 | −0.8 | 8.9 | |
| Vitamin C | EAR | mg | 60 | −301.9 | 0.0 | |
| Vitamin D | EAR | mcg | 10 | 5.7 | 95.8 | At Risk |
| Vitamin E | EAR | mg | 12 | 5.7 | 98.0 | At Risk |
| Vitamin K | AI | mcg | 90 | −10.7 | 67.5 | At Risk |
| Zinc | EAR | mg | 6.8 | −0.3 | 45.5 | At Risk |

TABLE 7
Nutritional Analysis of the 5:2 ADF Regimen for Males.

| | | | EAR/ | | | |
|---|---|---|---|---|---|---|
| | DRI | | AI | Mean | Percent | Assess- |
| Nutrient | Type | Units | Value | Inadequacy | Inadequate | ment |
| Total fat | EAR | g | 20 | −19.7 | 0.0 | |
| Fiber | AI | g | 34.8 | 11.2 | 97.5 | At Risk |
| Calcium | EAR | mg | 860 | −227 | 24.4 | At Risk |
| Copper | EAR | mcg | 0.7 | −0.7 | 0.0 | |
| Food folate | EAR | mcg | 320 | 17.4 | 60.4 | At Risk |
| Iron | EAR | mg | 6 | −7.2 | 0.9 | |
| Magnesium | EAR | mg | 346 | 13.8 | 63.4 | At Risk |
| Niacin | EAR | mg | 12 | −7.0 | 5.0 | |
| Potassium | AI | mg | 3000 | −1290 | 0.9 | |
| Riboflavin | EAR | mg | 1.1 | −1.0 | 2.0 | |
| Selenium | EAR | mcg | 45 | −36.5 | 1.1 | |
| Sodium | AI | mg | 1500 | −432 | 6.1 | |
| Vitamin A | EAR | mcg | 625 | −344 | 13.1 | |
| Vitamin B12 | EAR | mcg | 2. | −2.3 | 6.6 | |
| Vitamin B6 | EAR | mg | 1.22 | −1.5 | 0.1 | |
| Vitamin C | EAR | mg | 75 | −312.6 | 0.0 | |
| Vitamin D | EAR | mcg | 10 | 2.6 | 85.0 | At Risk |
| Vitamin E | EAR | mg | 12 | 5.5 | 98.1 | At Risk |
| Vitamin K | AI | mcg | 120 | 41.5 | 92.1 | At Risk |
| Zinc | EAR | mg | 9.4 | −0.01 | 57.0 | At Risk |

TABLE 8
Nutritional Analysis of the 5:2 ADF Regimen for Females.

| | | | EAR/ | | | |
|---|---|---|---|---|---|---|
| | DRI | | AI | Mean | Percent | Assess- |
| Nutrient | Type | Units | Value | Inadequacy | Inadequate | ment |
| Total fat | EAR | g | 18.4 | −15.6 | 2.5 | |
| Fiber | AI | g | 23.4 | 2.1 | 71.0 | At Risk |
| Calcium | EAR | mg | 880 | 29.1 | 54.8 | At Risk |
| Copper | EAR | mcg | 0.7 | −0.5 | 3.0 | |
| Food folate | EAR | mcg | 320 | 20.3 | 59.5 | At Risk |
| Iron | EAR | mg | 6.9 | −2.5 | 15.3 | |
| Magnesium | EAR | mg | 263 | −29.4 | 32.4 | At Risk |
| Niacin | EAR | mg | 11 | −2.7 | 21.1 | At Risk |
| Potassium | AI | mg | 2300 | −1337 | 0.8 | At Risk |
| Riboflavin | EAR | mg | 0.9 | −0.6 | 6.8 | |
| Selenium | EAR | mcg | 45 | −18.1 | 23.2 | At Risk |
| Sodium | AI | mg | 1500 | −92.0 | 45.1 | |
| Vitamin A | EAR | mcg | 500 | −207.0 | 31.8 | At Risk |
| Vitamin B12 | EAR | mcg | 2 | −0.5 | 40.4 | At Risk |
| Vitamin B6 | EAR | mg | 1.18 | −0.8 | 7.2 | |
| Vitamin C | EAR | mg | 60 | −314.1 | 0.0 | |
| Vitamin D | EAR | mcg | 10 | 5.7 | 94.9 | At Risk |
| Vitamin E | EAR | mg | 12 | 5.6 | 97.9 | At Risk |
| Vitamin K | AI | mcg | 90 | −12.3 | 63.9 | At Risk |
| Zinc | EAR | mg | 6.8 | −0.3 | 45.8 | At Risk |

In this Example, the nutrients identified as “At Risk” were similar by ADF regimen but differed for Males and Females. However, the magnitude of the nutrient gaps differed by regimen.

### Analysis: Distributions of Intakes

Tables 9-14 show the distributions of intakes for the nutrients of interest. The nutrients of interest are selected as those which have a risk of inadequacy large enough to be notable, and where the percentage of simulated diets which have inadequate intakes differs significantly from the baseline.

TABLE 9
Distribution of Intakes for Nutrients of Risk for Strict ADF Regimen, Males.

| Nutrient | Units | EAR/AI | RDA | Minimum | 2.5% | 50% | 97.5% | Maximum |
|---|---|---|---|---|---|---|---|---|
| Calcium | mg | 860 | 1000 | 240.8 | 435.9 | 1074.2 | 3050.1 | 4070.8 |
| Fiber | g | 34.8 | NA | 8.1 | 14.1 | 29.7 | 52.3 | 78.1 |
| Food Folate | mcg | 320 | 400 | 60.6 | 137.3 | 362.1 | 715.8 | 852.6 |
| Magnesium | mg | 346 | 416 | 160.0 | 229.4 | 461.1 | 668.7 | 769.1 |
| Vitamin D | mcg | 10 | 15 | 0.1 | 0.9 | 5.7 | 22.6 | 88.6 |
| Vitamin E | mg | 12 | 15 | 2.6 | 3.8 | 7.6 | 18.7 | 41.6 |
| Vitamin K | mcg | 120 | NA | 12.0 | 22.4 | 58.6 | 344.6 | 2231.2 |
| Zinc | mg | 9.4 | 11 | 2.8 | 4.4 | 10.3 | 25.0 | 48.7 |

TABLE 10
Distribution of Intakes for Nutrients of Risk for Strict ADF Regimen, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 170.2 | 302.5 | 720.7 | 2761.8 | 4063.1 |
| Fiber | g | 23.4 | NA | 5.9 | 11.2 | 25.9 | 46.9 | 62.1 |
| Food Folate | mcg | 320 | 400 | 59.8 | 119.7 | 293.2 | 709.5 | 862.7 |
| Magnesium | mg | 263 | 318 | 138.5 | 166.3 | 355.0 | 612.3 | 739.1 |
| Niacin | mg | 11 | 14 | 3.7 | 5.7 | 14.6 | 34.3 | 90.2 |
| Potassium | mg | 2300 | NA | 1754.4 | 2168.2 | 4370.7 | 8140.3 | 9481.9 |
| Selenium | mcg | 45 | 55 | 14.5 | 22.9 | 57.9 | 151.6 | 335.1 |
| Vitamin A | mcg | 500 | 700 | 29.3 | 123.1 | 404.5 | 2004.4 | 3008.2 |
| Vitamin B12 | mcg | 2.0 | 2.4 | 0.1 | 0.3 | 1.6 | 8.6 | 23.6 |
| Vitamin D | mcg | 10 | 15 | 0.0 | 0.2 | 0.8 | 17.5 | 29.3 |
| Vitamin K | mcg | 90 | NA | 7.5 | 17.1 | 48.0 | 397.9 | 747.7 |
| Zinc | mg | 6.8 | 8.0 | 2.0 | 2.9 | 6.6 | 17.1 | 43.2 |

TABLE 11
Distribution of Intakes for Nutrients of Risk for Modified ADF Regimen, Males.

| Nutrient | Units | EAR/AI | RDA | Minimum | 2.5% | 50% | 97.5% | Maximum |
|---|---|---|---|---|---|---|---|---|
| Calcium | mg | 860 | 1000 | 82.7 | 126.3 | 859.3 | 2440.0 | 3256.6 |
| Fiber | g | 34.8 | NA | 2.6 | 6.9 | 23.8 | 41.9 | 62.5 |
| Food Folate | mcg | 320 | 400 | 16.1 | 16.1 | 289.7 | 572.6 | 682.1 |
| Magnesium | mg | 346 | 416 | 69.0 | 119.6 | 368.9 | 535.0 | 615.2 |
| Vitamin D | mcg | 10 | 15 | 0.0 | 0.1 | 5.5 | 18.7 | 70.9 |
| Vitamin E | mg | 12 | 15 | 1.0 | 2.4 | 6.3 | 15.0 | 33.3 |
| Vitamin K | mcg | 120 | NA | 4.5 | 17.6 | 53.9 | 279.3 | 1784.9 |
| Zinc | mg | 9.4 | 11 | 1.8 | 1.8 | 8.4 | 20.1 | 38.9 |

TABLE 12
Distribution of Intakes for Nutrients of Risk for Modified ADF Regimen, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 5.9 | 85.1 | 761.6 | 2180.2 | 3250.4 |
| Fiber | g | 23.4 | NA | 0.0 | 3.6 | 22.0 | 36.8 | 49.7 |
| Food Folate | mcg | 320 | 400.0 | 0.0 | 25.6 | 303.0 | 553.0 | 690.1 |
| Magnesium | mg | 263 | 318 | 22.1 | 50.5 | 307.3 | 480.7 | 591.3 |
| Niacin | mg | 11 | 14 | 0.3 | 1.2 | 13.9 | 26.8 | 72.2 |
| Potassium | mg | 2300 | NA | 245.4 | 735.9 | 3691.5 | 6348.0 | 7585.5 |
| Selenium | mcg | 45 | 55 | 0.3 | 1.1 | 61.6 | 120.7 | 268.1 |
| Vitamin A | mcg | 500 | 700 | 0.0 | 12.0 | 680.9 | 1582.9 | 2444.5 |
| Vitamin B12 | mcg | 2.0 | 2.4 | 0.0 | 0.0 | 2.2 | 6.8 | 18.9 |
| Vitamin D | mcg | 10 | 15 | 0.0 | 0.0 | 3.5 | 14.5 | 23.5 |
| Vitamin K | mcg | 90 | NA | 0.0 | 1.6 | 67.1 | 326.8 | 598.1 |
| Zinc | mg | 6.8 | 8.0 | 0.2 | 0.4 | 7.2 | 13.4 | 34.5 |

TABLE 13
Distribution of Intakes for Nutrients of Risk for 5:2, Males.

| Nutrient | Units | EAR/AI | RDA | Minimum | 2.5% | 50% | 97.5% | Maximum |
|---|---|---|---|---|---|---|---|---|
| Calcium | mg | 860 | 1000 | 30.6 | 30.6 | 1095.1 | 2489.4 | 3256.6 |
| Fiber | g | 34.8 | NA | 1.1 | 3.3 | 26.8 | 43.0 | 59.8 |
| Food Folate | mcg | 320 | 400 | 4.5 | 4.5 | 355.3 | 589.8 | 682.1 |
| Magnesium | mg | 346 | 416 | 12.9 | 12.9 | 402.5 | 541.0 | 629.0 |
| Vitamin D | mcg | 10 | 15 | 0.0 | 0.0 | 6.7 | 19.3 | 70.9 |
| Vitamin E | mg | 12 | 15 | 0.1 | 0.3 | 6.9 | 16.3 | 36.7 |
| Vitamin K | mcg | 120 | NA | 0.6 | 1.4 | 65.7 | 295.5 | 1784.9 |
| Zinc | mg | 9.4 | 11 | 0.1 | 0.1 | 10.0 | 22.0 | 40.7 |

TABLE 14
Distribution of Intakes for Nutrients of Risk for 5:2, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 5.9 | 83.7 | 796.7 | 2136.6 | 3250.5 |
| Fiber | g | 23.4 | NA | 0.0 | 3.6 | 22.2 | 36.4 | 49.7 |
| Food Folate | mcg | 320 | 400 | 0.0 | 23.6 | 318.1 | 546.6 | 690.1 |
| Magnesium | mg | 263 | 318 | 22.1 | 49.9 | 312.7 | 475.2 | 591.3 |
| Niacin | mg | 11 | 14 | 0.3 | 1.1 | 14.2 | 26.2 | 72.2 |
| Potassium | mg | 2300 | NA | 245.4 | 745.1 | 3775.8 | 6250.9 | 7585.6 |
| Selenium | mcg | 45 | 55 | 0.3 | 1.0 | 60.7 | 120.4 | 268.1 |
| Vitamin A | mcg | 500 | 700 | 0.0 | 9.9 | 677.2 | 1569.5 | 2444.5 |
| Vitamin B12 | mcg | 2.0 | 2.4 | 0.0 | 0.0 | 2.2 | 6.8 | 18.9 |
| Vitamin D | mcg | 10 | 15 | 0.0 | 0.0 | 3.3 | 14.7 | 23.5 |
| Vitamin K | mcg | 90 | NA | 0.0 | 1.0 | 72.7 | 329.6 | 598.1 |
| Zinc | mg | 6.8 | 8.0 | 0.2 | 0.3 | 7.3 | 13.3 | 34.5 |

### Analysis: Amount of Inadequacy

Tables 15-20 show the amount below the EAR or AI for each percentile (2.5%, 50% and 97.5%) for the nutrients of interest. The nutrients of interest are selected as those which have a risk of inadequacy large enough to be notable, and where the percentage of simulated diets which have inadequate intakes differs significantly from the baseline. If the values are negative it means that the intake tends to be above the DRI.

TABLE 15
Amount of inadequacy of each “At Risk”
nutrient for Strict ADF for Males.

| | | | 2.5% | 50% | 97.5% |
|---|---|---|---|---|---|
| Nutrient | Units | EAR/AI | Gap | Gap | Gap |
| Calcium | mg | 860 | 424.1 | −214.2 | −2190.1 |
| Fiber | g | 34.8 | 20.7 | 5.1 | −17.5 |
| Food Folate | mcg | 320 | 182.7 | −42.1 | −395.8 |
| Magnesium | mg | 346 | 116.6 | −115.2 | −322.7 |
| Vitamin D | mcg | 10.0 | 9.1 | 4.3 | −12.6 |
| Vitamin E | mg | 12.0 | 8.2 | 4.4 | −6.7 |
| Vitamin K | mcg | 120 | 97.6 | 61.4 | −224.6 |
| Zinc | mg | 9.4 | 5.0 | −0.9 | −15.6 |

TABLE 16
Amount of inadequacy of each “At Risk”
nutrient for Strict ADF for Females.

| | | | 2.5% | 50% | 97.5% |
|---|---|---|---|---|---|
| Nutrient | Units | EAR/AI | Gap | Gap | Gap |
| Calcium | mg | 880 | 577.5 | 159.3 | −1881.8 |
| Fiber | g | 23.4 | 12.2 | −2.5 | −23.5 |
| Food Folate | mcg | 320 | 200.3 | 26.8 | −389.5 |
| Magnesium | mg | 263 | 96.7 | −92.0 | −349.3 |
| Niacin | mg | 11.0 | 5.3 | −3.6 | −23.3 |
| Potassium | mg | 2300 | 131.8 | −2070.8 | −5840.4 |
| Selenium | mcg | 45 | 22.1 | −12.9 | −106.6 |
| Vitamin A | mcg | 500 | 376.9 | 95.6 | −1504.4 |
| Vitamin B12 | mcg | 2.0 | 1.6 | 0.4 | −6.6 |
| Vitamin D | mcg | 10.0 | 9.8 | 9.2 | −7.5 |
| Vitamin K | mcg | 90 | 72.9 | 42.0 | −307.9 |
| Zinc | mg | 6.8 | 3.9 | 0.2 | −10.3 |

TABLE 17
Amount of inadequacy of each “At Risk”
nutrient for Modified ADF for Males.

| | | | 2.5% | 50% | 97.5% |
|---|---|---|---|---|---|
| Nutrient | Units | EAR/AI | Gap | Gap | Gap |
| Calcium | mg | 860 | 733.7 | 0.6 | −1580.1 |
| Fiber | g | 34.8 | 27.9 | 11.0 | −7.1 |
| Food Folate | mcg | 320 | 303.9 | 30.3 | −252.6 |
| Magnesium | mg | 346 | 226.3 | −22.9 | −189.0 |
| Vitamin D | mcg | 10.0 | 9.9 | 4.5 | −8.7 |
| Vitamin E | mg | 12.0 | 9.6 | 5.7 | −3.0 |
| Vitamin K | mcg | 120 | 102.4 | 66.1 | −159.3 |
| Zinc | mg | 9.4 | 7.6 | 1.0 | −10.7 |

TABLE 18
Amount of inadequacy of each “At Risk”
nutrient for Modified ADF for Females.

| | | | 2.5% | 50% | 97.5% |
|---|---|---|---|---|---|
| Nutrient | Units | EAR/AI | Gap | Gap | Gap |
| Calcium | mg | 880 | 794.9 | 118.4 | −1300.2 |
| Fiber | g | 23.4 | 19.8 | 1.4 | −13.4 |
| Food Folate | mcg | 320 | 294.3 | 17.0 | −233.0 |
| Magnesium | mg | 263 | 212.5 | −44.3 | −217.7 |
| Niacin | mg | 11.0 | 9.8 | −2.9 | −15.8 |
| Potassium | mg | 2300 | 1564.0 | −1391.6 | −4048.1 |
| Selenium | mcg | 45 | 43.9 | −16.6 | −75.7 |
| Vitamin A | mcg | 500 | 488.0 | −180.8 | −1082.9 |
| Vitamin B12 | mcg | 2.0 | 2.0 | −0.2 | −4.8 |
| Vitamin D | mcg | 10.0 | 10.0 | 6.5 | −4.5 |
| Vitamin K | mcg | 90 | 88.4 | 22.9 | −236.8 |
| Zinc | mg | 6.8 | 6.4 | −0.4 | −6.6 |

TABLE 19
Amount of inadequacy of each “At Risk”
nutrient for 5:2 for Males.

| | | | 2.5% | 50% | 97.5% |
|---|---|---|---|---|---|
| Nutrient | Units | EAR/AI | Gap | Gap | Gap |
| Calcium | mg | 860 | 829.4 | −235.1 | −1629.5 |
| Fiber | g | 34.8 | 31.5 | 8.0 | −8.2 |
| Food Folate | mcg | 320 | 315.5 | −35.3 | −269.8 |
| Magnesium | mg | 346 | 333.1 | −56.5 | −195. |
| Vitamin D | mcg | 10.0 | 10.0 | 3.3 | −9.3 |
| Vitamin E | mg | 12.0 | 11.7 | 5.1 | −4.3 |
| Vitamin K | mcg | 120 | 118.6 | 54.3 | −175.5 |
| Zinc | mg | 9.4 | 9.3 | −0.6 | −12.6 |

TABLE 20
Amount of inadequacy of each “At Risk”
nutrient for 5:2 for Females.

| | | | 2.5% | 50% | 97.5% |
|---|---|---|---|---|---|
| Nutrient | Units | EAR/AI | Gap | Gap | Gap |
| Calcium | mg | 880 | 796.3 | 83.3 | −1256.6 |
| Fiber | g | 23.4 | 19.8 | 1.2 | −13.0 |
| Food Folate | mcg | 320 | 296.4 | 1.9 | −226.6 |
| Magnesium | mg | 263 | 213.1 | −49.7 | −212.2 |
| Niacin | mg | 11.0 | 9.9 | −3.2 | −15.2 |
| Potassium | mg | 2300 | 1554.9 | −1475.8 | −3950.9 |
| Selenium | mcg | 45 | 44.0 | −15.7 | −75.4 |
| Vitamin A | mcg | 500 | 490.1 | −177.2 | −1069.5 |
| Vitamin B12 | mcg | 2.0 | 2.0 | −0.2 | −4.8 |
| Vitamin D | mcg | 10.0 | 10.0 | 6.7 | −4.7 |
| Vitamin K | mcg | 90 | 89.0 | 17.3 | −239.6 |
| Zinc | mg | 6.8 | 6.5 | −0.5 | −6.5 |

### Recommendations

In order to construct dietary recommendations, we consider the upper and lower bounds of the intake recommendations to identify nutrient targets, without exceeding the UL (see FIGS. 1-4 for examples for calcium and folate). The “Amount of Inadequacy” is used to generate the dietary recommendations. First, we take the difference between the RDA, or the AI if no RDA is available, and the 2.5% intake percentile as the upper and the difference with the 97.5% intake percentile as the lower. All recommendations are based on an energy level for the diet of 2000 kcal. The upper end of the recommendation is capped to ensure that the recommendation does not exceed the UL.

We then employ a computerized Diet Simulator. By making certain assumptions, the Diet Simulator can provide a reasonable estimate of the nutritional inadequacies/gaps and thus provide recommendations on how to close those gaps from nutrients, foods, beverages, and/or dietary supplements, and their combinations, without requiring undue effort or input from the user. In this Example, assumptions included the following:
The individual eats a typical meal pattern (e.g., breakfast, lunch, dinner and one snack per day),
The individual eats typical food and beverage combinations for those meals and snacks (e.g., meals and snacks as reported in NHANES), and
The individual intends to follow a well-balanced diet in accordance with dietary recommendations (e.g., the USDA Healthy Eating Pattern).

The food composition database used by the Diet Simulator contains the nutrient content, including macro- and micro-nutrients for each food per 100 g and per representative serving sizes. In this example, the USDA Food Data Central databases are used to identify foods and beverages containing sufficient levels of the nutrients identified as “At Risk” in the preceding steps. The Diet Simulator is also linked to a recipe database so that recipes which contain food sources of the nutrients “At Risk” can be identified.

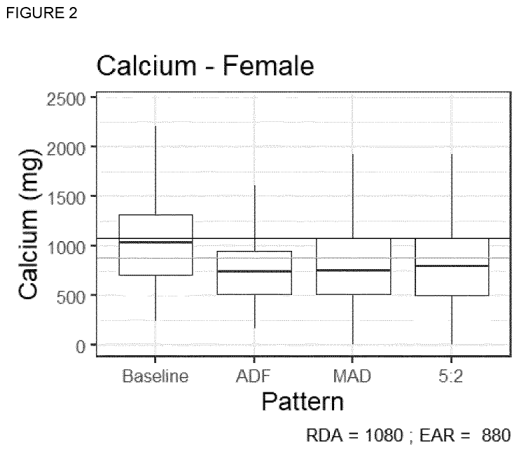

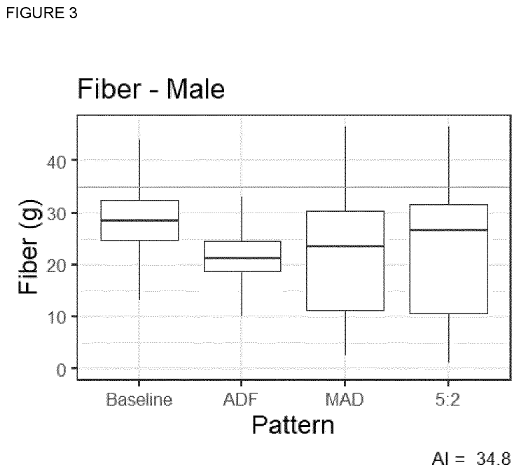

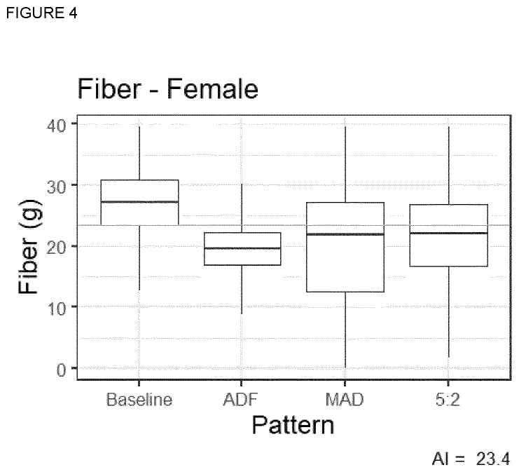

In this example, we have selected only two of these nutrients to exemplify recommendations. Thus, for e.g., fiber and calcium that were identified as nutrients of need for all of the ADF regimens. See FIGS. 1 to 4. FIG. 1 shows calcium intake ranges comparing simulated baseline intakes with Strict ADF, Modified ADF and 5:2 for males. FIG. 2 shows calcium intake ranges comparing simulated baseline intake with Strict ADF, Modified ADF and 5:2 for females. FIG. 3 shows fiber intake ranges comparing simulated baseline intakes with Strict ADF, Modified ADF and 5:2 for males. FIG. 4 shows fiber intake ranges comparing simulated baseline intakes with Strict ADF, Modified ADF and 5:2 for females.

Examples, such as the following dietary recommendations would be generated to accompany feedback on each of the ADF diets. In a similar manner, dietary recommendations for all of the nutrients identified as “At Risk” could be generated. As an illustration, we provide examples for calcium and dietary fiber as follows:

Calcium:

Calcium could be recommended from food sources. For example, someone specifying a vegetarian diet may receive a recommendation for soybean curd (tofu) cubes; 1 cup (248 g) provides 275 mg of calcium. Similarly, a recipe for tofu with mixed vegetables, including broccoli and carrots with a soy-based sauce could be recommended; 2 cups (434 g) provides 286 mg of calcium.

Calcium could be recommended from beverages. For example, someone specifying a flexitarian diet may receive a recommendation for low-fat milk; 1 cup (246 g) of low-fat milk contains 310 mg of calcium.

Calcium could be recommended as a dietary supplement. Non-limiting examples of suitable forms of calcium include one or more calcium salts, such as calcium acetate, calcium carbonate, calcium chloride, calcium citrate, calcium gluconate, calcium lactate or mixtures thereof.

The amount of additional calcium recommended would depend on the needs identified in the simulations. For example, the Strict ADF for men showed a deficit of 424 mg of calcium per day (Table 15). This amount could be provided by 336 g of milk, or about 1.4 cups.

Fiber

Fiber could be recommended from food sources. For example, someone specifying a Vegan diet may receive a recommendation for cooked oatmeal; 1 cup (234 g) provides 4 mg of dietary fiber. Similarly, a recipe for oatmeal raisin cookies could be recommended; 2 cookies (48 g) provides 1.6 g of fiber.

Fiber could be recommended from dietary supplements. Non-limiting suitable forms of fiber supplements include those containing wheat dextrin, methylcellulose, psyllium husk, polycarbophil, guar fiber, glucomannan or β-glucans.

1. A computer implemented method or AI based system to identify and/or quantify individual nutritional inadequacies in an individual following or planning to follow an Alternate Day Fasting (ADF) regimen, comprising the steps of:
i) Identifying the individual's nutritional needs and/or dietary rules,
ii) Comparing the individual's nutritional needs and/or dietary rules with the nutrients provided by an ADF diet, and
iii) Performing analyses to identify and quantify potential nutritional inadequacies.

2. A computer implemented method or system according to claim 1 wherein the Alternate Day Fasting regimen is selected from the group consisting of strict ADF, Modified ADF and 5:2 protocol.

3. A computer implemented method or system according to claim 1, wherein the individual's nutritional needs are based on at least one characteristic selected from the group consisting of age, gender, height, weight, physical activity, lifestyle and medical condition.

4. A computer implemented method or system according to claim 1, wherein the dietary rules are based on individual's specific dietary restrictions.

5. A computer implemented method or system according to claim 1, wherein the ADF diet of step ii) is a simulated ADF diet.

6. A computer implemented method or AI-based system to mitigate nutrient inadequacies in an individual following or planning to follow an Alternate Day Fasting regimen, said method comprising:
i) identifying nutritional inadequacies in the context of a defined ADF diet,
ii) providing specific dietary recommendations and/or nutritional solutions to mitigate the identified nutritional inadequacies in said individual, and
iii) providing recommendations on lifestyle.

7. A computer implemented method or system according to claim 6, wherein the method to identify nutritional inadequacies is a computer implemented method or AI based system to identify and/or quantify individual nutritional inadequacies in an individual following or planning to follow an Alternate Day Fasting (ADF) regimen, comprising the steps of:
i) Identifying the individual's nutritional needs and/or dietary rules,
ii) Comparing the individual's nutritional needs and/or dietary rules with the nutrients provided by an ADF diet, and
iii) Performing analyses to identify and quantify potential nutritional inadequacies.

8. A computer implemented method or AI-based system according to claim 6, wherein said method or system comprises diet recommendations, menu recommendations and/or recipe recommendations.

9. A computer implemented method or AI-based system according to claim 6, wherein said method provides recommendations for food or nutrients selected from the group consisting of nutrients, foods and beverages, dietary supplements, combinations of foods and beverages as menu recommendations and/or recipe recommendations, and combinations thereof.

10. A method of mitigating nutrient inadequacies in an individual following or planning to follow an Alternate Day Fasting regimen, said method comprising:
i) Identifying the individual's nutritional needs and/or dietary rules,
ii) Comparing the individual's nutritional needs and/or dietary rules with the nutrients provided by an ADF diet,
iii) Performing analyses to identify and quantify potential nutritional inadequacies, and
iv) Providing specific dietary recommendations and/or nutritional solutions to mitigate the identified nutritional inadequacies in said individual.

11-13. (canceled)
